(19) United States
(12) Patent Application Publication
(10) Pub. No.: US 2005/0245886 A1
(43) Pub. Date: Nov. 3, 2005

(54) Anti-ocular chamber collapse sleeve

(76) Inventors: Terence M. Devine, Athens, PA (US); Michael W. Geiger, Creve Coeur, MO (US)

Correspondence Address: Bausch & Lomb Incorporated, One Bausch & Lomb Place, Rochester, NY, 14604-2701, US

(21) Appl. No.: 11/105,153

(22) Filed: Apr. 13, 2005

Related U.S. Application Data
(60) Provisional application No. 60/566,500, filed on Apr. 29, 2004.

(51) Int. Cl. A61M 25/00

(52) U.S. Cl. 604/264; 604/22

(57) ABSTRACT

A phacoemulsification sleeve 10 includes a shaft member 14 attached to a hub portion 18 for attachment to a distal end of a handpiece 12. The sleeve 10 is in communication with an irrigation supply line 22 of the handpiece 12. The hub portion 18 is preferably formed of a flexible material and has an internal volume sufficiently large to prevent ocular chamber collapse upon occurrence of post-occlusion surge.

Priority is hereby claimed in the present nonprovisional application to Provisional Application Ser. No. 60/566,500 filed Apr. 29, 2004, in accordance with 37 CFR 1.78(a)(4).

### BACKGROUND OF THE INVENTION

1. Field of the Invention

The present invention is directed to an ophthalmic surgical handpiece sleeve for directing irrigation fluid from the handpiece to a surgical site. More specifically, the present invention is directed to a surgical sleeve having a sufficient internal volume to prevent ocular tissue damage upon an occurrence of post-occlusion surge during surgery.

2. Description of Related Art

Typically, present day eye surgery is performed in closed systems, which maintain an internal pressure of the ocular globe during surgery. Such surgery typically combines irrigation and aspiration to carry away excised tissue from the surgical site and to flush out or clean the surgical site. Typically, the surgeon controls the pressure of the irrigation and the aspiration so that a desired intraocular pressure is maintained during the surgical procedure.

If aspiration suction is too strong, it may damage epithelial cells or otherwise traumatize ocular tissue, including tearing the capsular bag. On the other hand, too high an irrigation pressure may increase the intraocular pressure sufficiently to traumatize ocular tissue.

The balance between aspiration vacuum levels and irrigation pressure levels are typically maintained by the surgeon through manipulation of aspiration pump performance and irrigation bottle height or the amount of gas-forced infusion pressure applied to an irrigation source.

One dangerous event during ocular surgery is typically referred to post-occlusion surge. This typically occurs when a tissue fragment becomes lodged in, for instance a phacoemulsification needle, and the aspiration pump continues to apply suction to the needle. However, because the ocular tissue is occluding the aspiration line no fluid or tissue is being aspirated through the aspiration line, which can cause portions of the aspiration line to collapse. Then upon the ocular tissue becoming dislodged from the aspiration line a sudden surge in vacuum pressure can be experienced in the ocular globe due to the rebounding of the aspiration lines.

Once a post-occlusion surge occurs a large volume of liquid in a very short period of time may be sucked from the ocular globe. The fluids may be sucked from the ocular globe faster than can be replaced by the irrigation fluid from the relatively rigid irrigation line and bottle. This can cause significant damage to ocular tissue during the collapse and cause highly undesirable complications to the surgery. If a sufficient volume of irrigation fluid could be transmitted into the ocular globe upon the occurrence of post-occlusion surge in a timely manner, ocular chamber collapse and hence ocular tissue damage could be prevented.

However, typical prior art systems cannot deliver such an amount of irrigation fluid in a sufficiently short period of time due to the fluid dynamics of the irrigation line. The irrigation line has too much resistance over too great of a distance to supply the required amount of fluid in the necessary short time.

At least one prior art patent, U.S. Pat. No. 4,841,984 to Armeniades, et al. entitled Fluid-Carrying Components of Apparatus for Automatic Control of Intraocular Pressure attempted to deal with this issue by providing a compliance chamber or damping devices at the proximal end of a surgical handpiece or in an irrigation line to the rear of an irrigation handpiece. These configurations are somewhat cumbersome for the surgeon to use and are still some distance away from the surgical site. The teachings of Armeniades, et al. are hereby incorporated into this specification by reference.

Therefore, it would be advantageous to provide a volume of fluid as close to the surgical site as possible to be delivered to the surgical site upon the occurrence of a post-occlusion surge in order to prevent damage to ocular tissue. Further advantages may be obtained by ensuring that the volume of fluid is contained in an area, such as a holding chamber or sleeve that deforms more readily than the ocular globe.

### BRIEF DESCRIPTION OF THE DRAWINGS

FIG. 1 is a cut-away perspective view of a phacoemulsification handpiece with a sleeve in accordance with the present invention; and

FIG. 2 is an alternative embodiment of a sleeve in accordance with the present invention.

### DETAILED DESCRIPTION OF THE PREFERRED EMBODIMENT

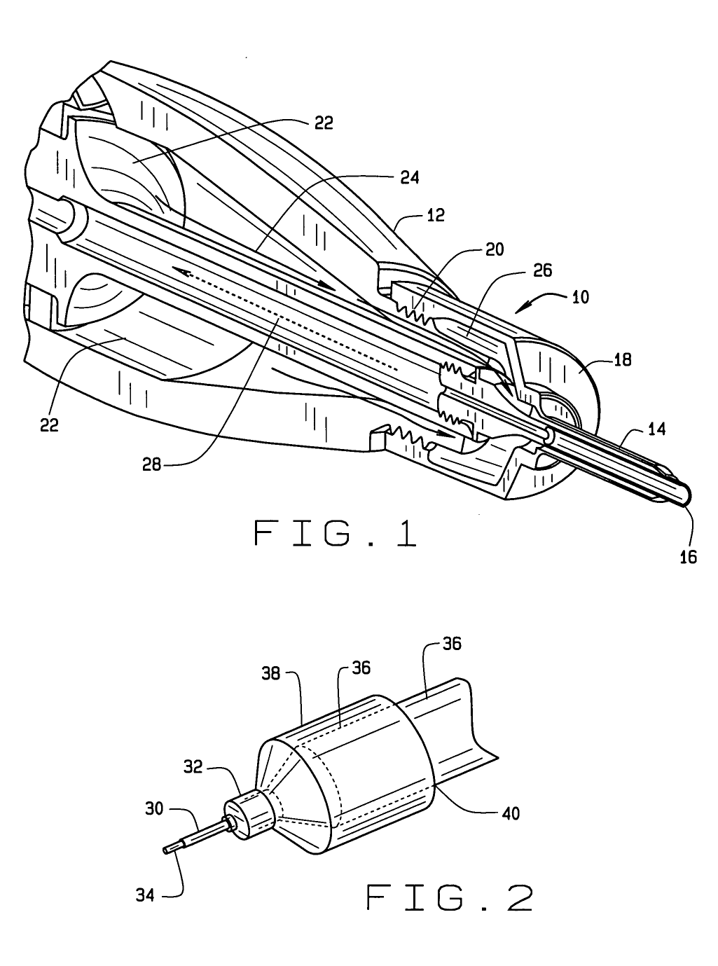

FIG. 1 shows a cut-away perspective view of a phacoemulsification handpiece with a sleeve in accordance with the present invention. A phacoemulsification sleeve, shown generally at 10, is attached to a phacoemulsification handpiece 12. Sleeve 10 includes a shaft member 14 for surrounding a phacoemulsification needle 16 as shown, a hub portion 18 is integral to and attached to the shaft member 14 for attachment to a distal end of a phacoemulsification handpiece 12. Typically this attachment is via threads 20 so that precise location of shaft member 14 may be made relative to needle 16. As can be seen, hub portion 10 and shaft member 14 are in communication with an irrigation supply line 22 of handpiece 12.

In use, irrigation fluid flows into sleeve 10 in the direction of arrows 24 and is eventually delivered to a surgical site (not shown). Hub portion 18 is preferably formed of a flexible material, such as silicone or other material that is sufficiently flexible and thin so that the hub 18 collapses upon a surge condition more readily than the ocular chamber. Hub 18 has an internal volume, shown generally at 26, which is sufficiently large to prevent ocular chamber collapse upon occurrence of a post-occlusion surge. Preferably, internal volume 26 has a volume of 5 cm³ to as much as 260 cm³ depending on the type of surgery to be performed and the size of hub portion 18 that can be tolerated by the surgeon. The minimum volume of hub 18 must be at least 0.5 cm³, which approximates the volume of a typical anterior chamber and lens capsule. Hub 18 is preferably molded in manufacture to shaft 14 as a single unit.

Upon a piece of ocular tissue occluding needle 16 becoming dislodged, the system will quickly aspirate fluid and tissue in the direction of dashed-arrow 28. By providing the inventive sleeve 10 with a sufficient internal volume of irrigation fluid immediately adjacent to surgical site, it is believed that significant damage that otherwise may be experienced upon such post-occlusion surge will be avoided or minimized by quickly delivering the volume of irrigation fluid available at internal volume 26 into the patient's eye. In addition, this internal volume of fluid may be transported to the surgical site in a very short time period because of its proximity to the surgical site and because hub 18 is made of a flexible material which allows it to collapse upon the experience of a sufficient vacuum draw.

FIG. 2 shows a partial perspective view of an alternate embodiment of a sleeve in accordance with the present invention. The sleeve of FIG. 2 includes a shaft member 30, a hub portion 32 which are similar to that described above where shaft member 30 surrounds a needle 34 that is attached to a handpiece 36. However, the sleeve of FIG. 2 also includes a large volume container 38 which surrounds a portion of handpiece 36 as shown; where the dash lines show the handpiece 36 contained within large volume container 38. In this way the sleeve of FIG. 2 can deliver a much larger volume of fluid to a surgical site in a rapid fashion as compared to the embodiment shown in FIG.1. It is noted that a fluid-tight seal needs to be provided between container 38 and handpiece 36 at the end of container 38 shown at 40. This seal may be of any known sealing mechanism, such as threads or press-fits, which allow for a fluid-tight seal to be maintained.

1. A phacoemulsification sleeve comprising:
a shaft member for surrounding a phacoemulsification needle;
a hub portion integral to the shaft member for attachment to a distal end of a phacoemulsification handpiece and in communication with an irrigation supply line of the handpiece; and
wherein the hub portion is formed of a flexible material that collapses more readily than the ocular chamber and has an internal volume sufficiently large to prevent ocular chamber collapse upon an occurrence of post-occlusion surge.

2. The sleeve of claim 1, wherein the sleeve is formed of silicone.

3. The sleeve of claim 1, wherein the sleeve has an internal volume of at least 0.5 cm³.

4. A surgical handpiece sleeve comprising:
a shaft member for surrounding a distal end of the handpiece;
a hub portion attached to the shaft member for attachment to the handpiece;
wherein the hub portion and shaft member are in communication with an irrigation line of the handpiece for directing irrigation fluid to a surgical site and are formed of a flexible material that collapses more readily than the ocular chamber; and
wherein the sleeve has an internal volume sufficiently large to contain enough irrigation fluid to prevent ocular tissue damage upon an occurrence of post-occlusion surge.

5. The sleeve of claim 4, wherein the sleeve is formed of silicone.

6. The sleeve of claim 4, wherein the sleeve has an internal volume of at least 0.5 cm³.
